(19) United States
(12) Patent Application Publication
(10) Pub. No.: US 2023/0307098 A1
(43) Pub. Date: Sep. 28, 2023

(54) Technologies for Accessing and Transmitting Medical Data for Patients and Facilitating Treatments

(71) Applicant: GMSG Consulting LLC, Pharump, NV (US)

(72) Inventor: Gerald Gryschuk, Pharump, NV (US)

(21) Appl. No.: 17/702,604

(22) Filed: Mar. 23, 2022

(51) Int. Cl. G16H 10/60; G16H 40/67; G16H 20/00

(52) U.S. Cl. CPC G16H 10/60; G16H 40/67; G16H 20/00

(57) ABSTRACT

Systems and methods for managing treatments for patients are described. According to certain aspects, an electronic device retrieves and stores treatment data associated with a given patient. A console device enables selection of the patient and requests the electronic device for up-to-date treatment data for that patient. Further, the console device causes a medical device to administer treatment to the patient according to the treatment data. A set of “as treated” data associated with the treatment is sent to the electronic device, which transmits the as treated data to the server for storage thereon.

### FIELD

The present disclosure is directed to managing the administration of medical treatments to patients. More particularly, the present disclosure is directed to platforms and technologies for storing and retrieving treatment plans associated with the administration of medical treatments to patients.

### BACKGROUND

Innovations in the medical space continue to result in improved and more efficient disease diagnoses and patient treatments, among other things. For example, certain companies manufacture linear accelerators that treat cancer and other medical conditions with radiotherapy, radiosurgery, proton therapy, brachytherapy, and other treatments. Generally, these treatment devices support software that controls operation of the devices according to treatment plans that are specific to the care of individual patients. For example, different patients need different degrees and durations of radiation to treat different types of cancer, as determined by physicians.

Current device and treatment implementations rely on network connections to back-end servers that store data associated with treatment plans. In particular, when a physician decides on a treatment for a medical device to administer to a patient, a treatment plan is created and sent to a back-end server for storage. For a patient to receive treatment, a console or terminal at the treatment site interfaces with the back-end server to retrieve the treatment plan and sends the treatment plan to the medical device which administers the treatment accordingly.

However, problems arise when a connection to the back-end server is unavailable. In these situations, any available treatment data which typically lacks all relevant data must be manually exported to a manually-preconfigured storage location which must be constantly maintained to ensure that sufficient storage is always available. Additionally, outdated or stale data must be manually removed and, in the event that treatment data is actually used for treatment, this data must be manually uploaded to the back-end server for storage when the connection is available.

Therefore, there is an opportunity for systems and methods to load and save relevant treatment data in near constant time regardless of a network speed or connection status, physical distance between a treatment device and data storage components, and/or operating speed and status of relevant information systems and back-end storage servers.

### SUMMARY

In an embodiment, a computer-implemented method in an electronic device of managing treatment for patients is provided. The electronic device may be communicatively connected to a console device that is communicatively connected to a medical device that administers treatment to the patients. The computer-implemented method may include: retrieving, by a processor from a server via a first network connection, treatment data associated with a patient; storing, in a memory of the electronic device, the treatment data associated with the patient; receiving, by the processor from the console device via a second network connection, an identification of the patient and a request for the treatment data associated with the patient; sending, by the processor to the console device via the second network connection, the treatment data, wherein the console device causes the medical device to administer the treatment to the patient according to the treatment data; and after the medical device administers the treatment to the patient according to the treatment data: receiving, by the processor from the console device via the second network connection, a set of data indicative of the treatment that was administered to the patient, storing, in the memory, the set of data, and transmitting, to the server, the set of data for storage at the server.

In another embodiment, an electronic device for managing treatment for patients is provided. The electronic device may be communicatively connected to a console device that is communicatively connected to a medical device that administers treatment to the patients. The electronic device may include: a transceiver configured to connect the electronic device to a server via a first network connection and to a console device via a second network connection; a memory; and a processor interfacing with the transceiver and the memory. The processor may be configured to: retrieve, from a server via the transceiver, treatment data associated with a patient, store, in the memory, the treatment data associated with the patient, receive, from the console device via the transceiver, an identification of the patient and a request for the treatment data associated with the patient, send, to the console device via the transceiver, the treatment data, wherein the console device causes the medical device to administer the treatment to the patient according to the treatment data, and after the medical device administers the treatment to the patient according to the treatment data: receive, from the console device via the transceiver, a set of data indicative of the treatment that was administered to the patient, store, in the memory, the set of data, and transmit, to the server via the transceiver, the set of data for storage at the server.

Further, in an embodiment, a non-transitory computer-readable storage medium having stored thereon a set of instructions executable by at least one processor of an electronic device is provided. The non-transitory computer-readable storage medium may be for managing treatment for patients, where the electronic device is communicatively connected to a console device that is communicatively connected to a medical device that administers treatment to the patients. The instructions may include: instructions for retrieving, from a server via a first network connection, treatment data associated with a patient; instructions for storing, in a memory, the treatment data associated with the patient; instructions for receiving, by a processor of the electronic device from the console device via a second network connection, an identification of the patient and a request for the treatment data associated with the patient; instructions for sending, by the processor to the console device via the second network connection, the treatment data, wherein the console device causes the medical device to administer the treatment to the patient according to the treatment data; and instructions for, after the medical device administers the treatment to the patient according to the treatment data: receiving, from the console device via the second network connection, a set of data indicative of the treatment that was administered to the patient, storing, in the memory, the set of data, and transmitting, to the server, the set of data for storage at the server.

### BRIEF DESCRIPTION OF THE FIGURES

FIG. 1A depicts an overview of components and entities in conventional implementations of treatment systems, in accordance with some embodiments.

FIG. 1B depicts an overview of components and entities configured to facilitate the described systems and methods for managing treatment of patients, in accordance with some embodiments.

FIG. 2 illustrates a signal diagram of the functionalities for managing treatment of patients, in accordance with some embodiments.

FIGS. 3A-3C depict example interfaces associated with managing treatment of patients, in accordance with some embodiments.

FIG. 4 illustrates an example flow diagram of managing treatment for patients, in accordance with some embodiments.

FIG. 5 is a hardware diagram of an example server and an example electronic device configured to facilitate the described techniques, in accordance with some embodiments.

### DETAILED DESCRIPTION

The present embodiments may relate to, inter alia, platforms and technologies for managing medical treatment for patients. According to certain aspects, an electronic device may be connected to a console device that may be connected to a medical device that may administer treatment or therapy (e.g., radiation therapy) to patients. The electronic device may retrieve, from a remote server, treatment data associated with a given patient and store the treatment data in memory. The console device may request the treatment data for the patient from the electronic device, which may provide the treatment data to the console device. In turn, the console device may cause the medical device to administer the treatment or therapy to the patient according to the treatment data.

After the medical device administers the treatment, the console device may send, to the electronic device, a set of data indicative of the treatment or therapy that was administered, and the electronic device may store the set of data in memory as well as transmit the set of data to the server for storage at the server. According to embodiments, in the event of a failed connection between the electronic device and the server, the electronic device may store the set of data and transmit the set of data to the server once the connection is restored.

The systems and methods offer numerous benefits. In particular, the systems and methods automatically cache all relevant treatment data to be used for patient treatments on a per-medical device basis for a selectable period of time (e.g., a day, week, month, etc.). Further, the systems and methods automatically maintain updates to treatment data should the data be modified during the life cycle of a patient's treatment. Additionally, during the transmission of “as delivered” treatment data and imaging information to a remote server, the systems and methods automatically store this data and information, thus enabling a secondary archive which may be subsequently accessed for subsequent operations. Specifically, the systems and methods are able to recover treatment and imaging data which may otherwise be lost in the event of a connection failure to a remote server. Further, the systems and methods are able to display data/information regarding completed treatments in an “offline” mode where this data/information is not normally able to be displayed.

Additionally, the systems and methods continuously attempt to transmit the “as delivered” treatment and imaging information to the remote server until successful or until a pre-defined number of failures occurs, versus a “one and done” attempt to save this data as exists in conventional implementations. An additional benefit is the systems and methods enable reinstating the automated saving of treatment and imaging information when access to the remote server is restored.

Further, the systems and methods enable automatic synchronization of treatment and imaging information, and provide an intuitive graphical user interface which displays patient and treatment-relevant information (e.g., photo, patient name, course name, plan name, patient birth data, age, scheduled appointment date and time, previous treatment history, etc.), thus enabling for selection of the appropriate patient and treatment plan to be delivered to the patient. Moreover, the systems and methods automatically delete stale data, thus ensuring there is always sufficient storage for new and updated data. It should be appreciated that additional benefits are envisioned.

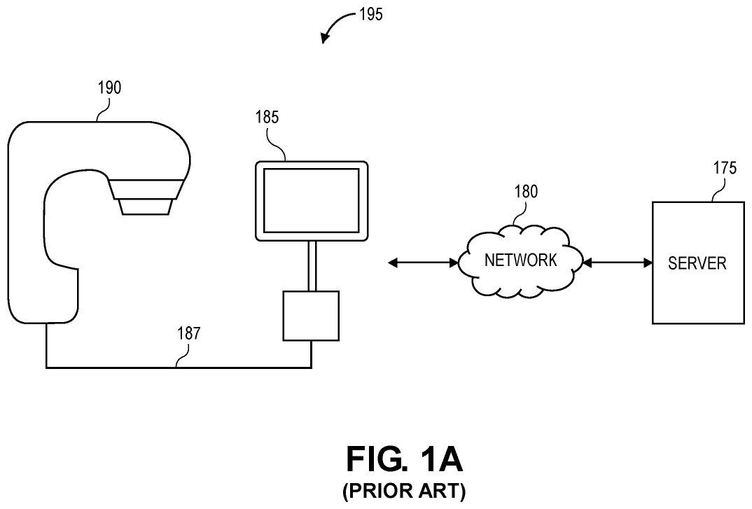

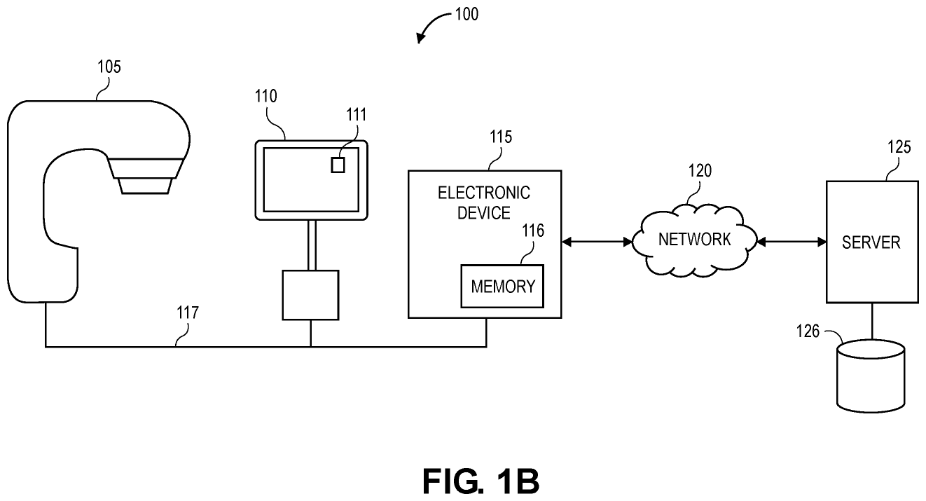

FIG. 1A illustrates an overview of a prior art system 195 of components configured to facilitate treatment to patients. In particular, the system 195 represents current implementations for managing operation of treatment devices to treat patients. The system 195 may include a console device 185 connected to a medical device 190 via a connection 187. The console device 185 may communicate with a server 175 via a network 180. According to embodiments, the medical device 190 may be configured to administer treatment to the patients, for example radiation therapy to treat cancer.

Generally, in using an application running on the console device 185, a user (e.g., a radiation therapist) will select an appointment (or a patient) to be treated on the medical device 190. Once the appointment is selected, the console device 185 communicates with the server 175 (e.g., using the Digital Imaging and Communications in Medicine (DICOM) protocol) to download treatment data for the selected patient to be treated. Treatment of the patient is controlled entirely by the console device 185 and related software running on additional computers (not depicted in FIG. 1A) known as on-board imager (OBI) and cone-beam computed tomography (CBCT) consoles. When the patient is ready for treatment, the user initiates the treatment from the console device 185 which transfers the appropriate treatment data to the medical device 190 which will cause the medical device 190 to operate according to the treatment data.

Once the entire treatment session has been completed, which for example may consist of one or more doses of radiation as well as performing “imaging events” such as taking a portal image (PV) or set of OBI images, the user “closes” the treatment session. The closing of the treatment session initiates the console device 185 to send the “as delivered” treatment and imaging information to the server 175 via the network 180, for example using the DICOM protocol.

In the event that the connection (i.e., the network 180) between the console device 185 and the server 175 is interrupted or unavailable, neither the saving of the “as delivered” treatment information nor the loading of the data for the next treatment can occur until the connectivity is restored between the console device 185 and the server 175.

It may be possible to allow treatments to continue on the medical device 190 in the event of loss of connectivity to the server 175, however this process is highly manual and requires significantly daily or even “on demand” exporting of treatment data to avail it for possible use in an “offline” mode (e.g., DICOM-RT mode). Specifically, a center must configure a physical storage location to be used to manually store exported treatment data, where the physical storage can be allocated on the console device 185 itself or on a separate computer or “file server”, as well as configured a storage location “per accelerator”. Additionally, the center must avail the storage location for access by other computers on the same network using a built-in common internet file system (CIFS) protocol. In the event the storage location is on the console device 185, the CIFS share is made available to be used as a network drive by any computer on the network. Typically, it is made available on only a small number of computers in the network due to security and usability concerns. If the storage location is on a separate server, typically the server 175 or another computer on the network, the storage location can be used on the console device 185, again using a CIFS protocol.

The benefit of placing this storage on the console device 185 itself and availing on the network is that it better ensures that the data will be available if network access is lost. It also ensures that the treatment data for a specific treatment device is localized to the physical location of the treatment device and its dedicated console device. For these reasons, if users choose to use an “offline” mode, they will use this method of exposing the storage on the network.

Further, in the process of exporting the treatment data to files, the data is stored with very limited information in the file name itself that can be used to select an appropriate treatment. For example, a filename might be ‘RP.1.2.456.435.65.5.9876542.105005.dcm’. The only “human interpretable” information is “RP” which identifies this file as a “plan file” used for treatment. While it is possible to export the data with a naming convention of, for example, “Patient ID+Object Suffix” where the “Patient ID” will be unique to a given patient, the Patient ID is rarely easily identifiable as belonging to a specific patient (e.g., “John Smith” may have a Patient ID of ‘R123456’) and this file naming convention may limit the types of files that can be loaded on the console device 185 and related software (e.g., the OBI software). To better manage this lack of information, it is recommended that users create directories “per patient” (e.g., “John Smith”) and subdirectories if multiple plans are to be treated (e.g., “ANT PELVIS”). A directory structure of this storage for a single patient may be, for example, “Z:\John Smith\ANT PELVIS”. This or a similar directory structure must be created for each new (unique) patient as well as new plans for each patient. Once the storage and directory structure methodology is defined, users must export the treatment data any time it changes, which is potentially weekly or daily, to keep it up to date just in case it needs to be used for treating in offline/DICOM-RT mode.

Moreover, depending on where the server 175 is physically located, which may be hundreds to thousands of miles away, the time taken to load treatment data and save the “as delivered” treatment information to the server 175 may take a considerable time portion of a single “treatment slot”. Generally, treatment slots are time periods used to help manage scheduling of treatments throughout the treatment day, and are typically ten (10), twelve (12), or fifteen (15) minutes long. Depending on the speed of the server 175, the server software, the physical network location and the distance between the console device 185 and the server 175, it can take less than a minute or up to five minutes to load or save data.

FIG. 1B illustrates an overview of a system 100 of components configured to facilitate the described systems and methods of managing treatment of patients. It should be appreciated that the system 100 is merely an example and that alternative or additional components are envisioned.

As illustrated in FIG. 1B, the system 100 may include a console device 110 and an electronic device 115. In embodiments, each of the console device 110 and the electronic device 115 may be any type of electronic device such as a mobile device (e.g., a smartphone), desktop computer, notebook computer, tablet, phablet, GPS (Global Positioning System) or GPS-enabled device, smart watch, smart glasses, smart bracelet, wearable electronic, PDA (personal digital assistant), pager, computing device configured for wireless communication, and/or the like. Further, each of the console device 110 and the electronic device 115 may include a user interface (e.g., a touchscreen and/or other component configured to present or display information to a user and/or receive inputs or selections from the user).

The system 100 may further include a medical device 105. According to embodiments discussed herein, the medical device 105 may be a linear accelerator configured to administer radiation therapy to the patients, for example to treat cancer. It should be appreciated that other types of medical devices are envisioned which may be configured to treat other types of diseases or ailments.

As illustrated in FIG. 1B, the medical device 105, the console device 110, and the electronic device 115 may be communicatively connected via a wired connection 117. Although FIG. 1B depicts the wired connection 117, it should be appreciated that different combinations of wired or wireless connections may exist between and among the medical device 105, the console device 110, and the electronic device 115.

The electronic device 115 may communicate with a server 125 via one or more networks 120. The server 125 may be associated with an entity that owns, operates, communicates with, and/or manages operation of the medical device 105, the console device 110, and/or the electronic device 115. In embodiments, the network(s) 120 may support any type of data communication via any standard or technology (e.g., GSM, CDMA, TDMA, WCDMA, LTE, EDGE, OFDM, GPRS, EV-DO, UWB, Internet, IEEE 802 including Ethernet, WiMAX, Wi-Fi, Bluetooth, and others).

The server 125 may be configured to interface with or support a memory or storage 126 capable of storing various data, such as in one or more databases or other forms of storage. According to embodiments, the storage 126 may store treatment data or treatment plans for patients, where the treatment data is associated with operation of the medical device 105.

The electronic device 115 may be configured with a memory 116 that may alternatively or additionally store treatment data for patients. According to embodiments, the electronic device 115 may retrieve the treatment data from the server 125 via the network(s) 120, for example using a unique identifier (UID), and store the retrieved data in the memory 116.

In embodiments, a single physical connection may be used on a network to which the console device 110 and the server 125 also have a connection. The communication between the electronic device 115 and the console device 110 or the server 125 may thus be separate logical connections but not necessarily separate physical connections. Alternatively, the electronic device 115 may support multiple independent physical connections, a first of which may be used to communicate with the console device 110 and a second of which may be used to communicate with the server 125.

The console device 110 may support and execute a treatment application 111 that may be configured to manage operation of the medical device 105 for various patients, where a user of the console device 110 may make selections associated with the treatment application 111 via a user interface. In particular, the treatment application 111 may display a listing of appointments for a set of patients, where the user may select an appropriate appointment as well as select to begin a treatment for the patient having that appointment. It should be appreciated that the electronic device 115 may alternatively or additionally support and execute the treatment application 111, or otherwise interface with the treatment application 111.

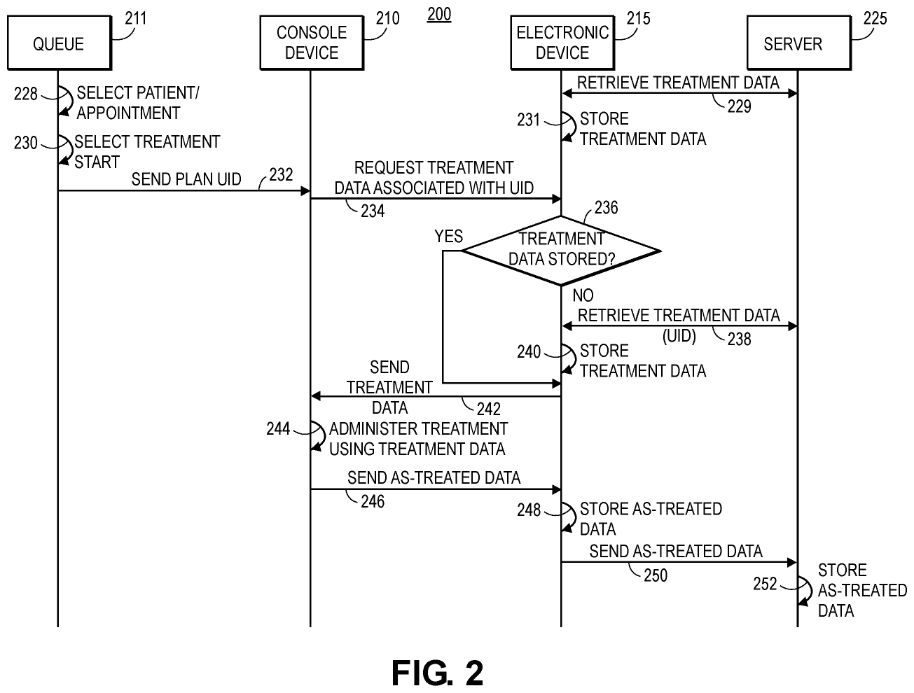

When the user selects to begin a treatment for a patient, the console device 110 may retrieve, from the electronic device 115, treatment data associated with the selected treatment. Additionally, the console device 110 may cause the medical device 105 to begin administering the treatment to the patient. After the medical device 105 administers the treatment to the patient, the console device 110 may send information indicative of the treatment to the electronic device 115, which may store this information in the memory 116 and/or transmit the information to the server 125 via the network(s) 120, for storage in the storage 126. These functionalities are described in further detail with respect to FIG. 2.

Although depicted as a single server 125 in FIG. 1B, it should be appreciated that the server 125 may be in the form of a distributed cluster of computers, servers, machines, or the like. In this implementation, the entity may utilize the distributed server(s) 125 as part of an on-demand cloud computing platform. Accordingly, when the electronic device 115 interfaces with the server 125, the electronic device 115 may actually interface with one or more of a number of distributed computers, servers, machines, or the like, to facilitate the described functionalities.

FIG. 2 illustrates a signal diagram 200 associated with managing treatment for patients. The signal diagram 200 may include a queue 211, a console device 210, an electronic device 215, and a server 225. According to embodiments, the server 225 may be the same as or similar to the server 125 as discussed with respect to FIG. 1B; the electronic device 215 may be the same as or similar to the electronic device 115 as discussed with respect to FIG. 1B; and the console device 210 may be the same as or similar to the console device 110 as discussed with respect to FIG. 1B. Further, the queue 211 may represent the treatment application 111 as discussed with respect to FIG. 1B. According to embodiments, the queue 211 may be incorporated into and executed by the console device 210, or may be part of an electronic device separate from the console device 210. Further, the queue 211 may support a user interface, which may be a component of the console device 210 and which may be configured to display or present content and via which a user may make selections.

The signal diagram 200 may begin when the queue 211 receives (228), via the user interface, a selection for a patient and/or an appointment. It should be appreciated that the patient and/or appointment may be selected automatically, such as by interfacing with a schedule that lists appointments on specific dates and times. Further, it should be appreciated that the queue 211 may display a listing of appointments, a listing of patients, information associated with the patients, information associated with treatments for the patients, and/or other information.

After the patient and/or appointment has been selected, the queue 211 may receive (230), via the user interface, a selection to start the treatment. Generally, a given treatment plan may have a unique identifier (UID) that uniquely identifies the treatment plan across multiple countries, sites, vendors, and equipment. For example, a UID may be used within the context of the DICOM Standard where the UID may be a registered value as defined by ISO 9834-1, and may be composed of two parts, an <org root> and a <suffix>. It should be appreciated that different formats and standards for the UID are envisioned.

Before, concurrent with, or after the functionalities of 228 and/or 230 are performed, the electronic device 215 may retrieve (229), from the server 225, treatment data for a set of patients, which may or may not include the patient corresponding to the selection of 228. Further, the electronic device may store (231) the retrieved treatment data in local memory. According to embodiments, the electronic device 215 may consistently query the server 225 for updated treatment data associated with the set of patients (e.g., every fifteen (15) minutes, two hours, etc.), and may overwrite stored treatment data with updated treatment data, to increase a likelihood that the electronic device 215 stores the most up-to-date treatment data.

The queue 211 may send (232) or avail the UID corresponding to the treatment (and/or to the patient) to the console device 210. After receiving or accessing the UID corresponding to the treatment, the console device 210 may request (234), from the electronic device 215, treatment data associated with the UID.

According to embodiments, the electronic device 215 may or may not store up-to-date treatment data associated with the UID. Generally, any object (e.g., a DICOM object such as a treatment plan, image, etc.) must have a UID that is globally unique. If anything in that object changes (e.g., if the treatment plan changes), then this is considered new data and a new object which has a different globally-unique UID is created and provided to the electronic device 215. Therefore, because the UID is always globally unique for any given object, the electronic device 215 is able to determine whether it stores the requested objects. In particular, if the electronic device 215 stores up-to-date treatment data associated with the UID, the electronic device 215 may link, within a relational database or other type of storage, the treatment data (and any related objects) with the UID.

Using the UID, the electronic device 215 may determine (236) whether the treatment data is locally stored, either by memory incorporated into the electronic device 215 or from memory/a database that may be locally accessed by the electronic device 215. If the treatment data is not locally stored (“NO”), the electronic device 215 may retrieve (238) treatment data corresponding to the UID from the server 225. According to embodiments, the treatment data retrieved from the server 225 may represent up-to-date treatment data that was previously not provided to or retrieved by the electronic device 215. After retrieving the treatment data, the electronic device 215 may store (240) the treatment data in memory. Processing may then proceed to (242).

If the treatment data is locally stored (“YES”), the electronic device may access the treatment data and send (242) the treatment data to the console device 210. The console device 210 may cause the treatment to be administered (242) to the patient using the treatment data received from the electronic device 215. In embodiments, the console device 210 may interface with a medical device (e.g., the medical device 105 as discussed with respect to FIG. 1) to cause the medical device to administer treatment to the patient according to the treatment plan.

Concurrently with or following administration of the treatment, the console device 210 may send (246) as-treated data associated with the treatment to the electronic device 215. In particular, the data may indicate that the treatment was administered to the patient, either partially or fully, and may include details of the administered treatment, including any images captured by the medical device. It should be appreciated that the data may include additional information. The electronic device 215 may store (248) the as-treated data in memory. Because the electronic device 215 may be physically local to the console device 210, the time that the electronic device 215 takes to store this as-treated data is minimal, thus availing the console device 210 to resume operation to treat a subsequent patient.

Additionally or alternatively, the electronic device 215 may send (250) the as-treated data to the server 225, which may store (252) the as-treated data. Accordingly, either or both of the server 225 and the electronic device 215 may store an up-to-date data associated with treatment(s) that a given patient has undertaken. According to embodiments, the electronic device 215 may request that the server 225 store the data in the background or as part of a separate thread of operation, thus availing the console device 210 to resume operation to treat a subsequent patient. If there is an error in communication between the electronic device 215 and the server 225, the electronic device 215 may continuously attempt to transmit the as-treated data to the server 225 until a connection is restored, until a timeout condition occurs, or according to another condition.

In the event that access to the server 225 is interrupted, the electronic device 215 may report an “error condition”, such as via an electronic message or notification to a user (e.g., a radiation therapist) and will fail to respond to the request for the treatment data from the console device 210. In response, the user may select to treat the patient in an offline mode (e.g., DICOM-RT) on the console device 210 with the benefit that the electronic device 215 has automatically and locally stored treatment data for the patient (and other patients) as well as data associated with treatments that occurred with the electronic device 215 in “normal” operation (i.e., with access to the server 225).

According to embodiments, the electronic device 215 may periodically or consistently query the server 225 for new and updated treatment and/or patient data, as well as data related to treatments/appointments scheduled to be delivered and other data relevant to patients such as, for example, transportation information (i.e., information as to how patients will be transported to the treatment center), patient demographics including photos, contact lists, medical information relevant to treatment, identification of primary and other physicians, and/or other data. The period of time between these automated queries may be default or specified by a user, and may for example be every fifteen (15) minutes to minimize load on the server 225 while maximizing the probability that the most up-to-date treatment and patient-related data is stored on the electronic device 215.

While the electronic device 215 operates in an offline mode, a user may review, via a user interface of the electronic device 215 or the console device 210, relevant appointment information and select a patient to be treated and a treatment plan for that patient. After the user makes the selections, treatment data specific to the selected patient and plan may be copied to a “staging area”, which may be configured to be mounted by the console device 210. The file naming convention for the treatment data may be the same as that of data that is exported directly from the server 225 (e.g., ‘RP.1.2.456.435.65.5.9876542.105005.dcm’) because there may be only a single plan file staged at the time of treatment. Thus, the user may be guaranteed to be selecting the treatment plan corresponding to that would be selected in a normal mode of operation. At this point, the electronic device 215 or the console device 210 may notify the user that the treatment plan has been staged for use on the console device 210. According to embodiments, the user may then select a treatment plan for offline treatment via the console device 210. In particular, the user may select the file corresponding to the treatment plan, causing the file and other relevant files in the same directory (e.g., reference images) to be loaded by the console device 210.

After the console device 210 reflects that the treatment is complete, the user may acknowledge, via a user interface of the console device 210, that the treatment is complete, at which point the “as-treated” data stored to a file service location may be copied internally within the electronic device 215 (e.g., to a location that may be inaccessible to any other device on a connected network) for later storage to the server 225 when connectivity to the server 225 has been restored. At that point, all files may be deleted from the file service location, thus ensuring that no conflicting information remains to await selection of a subsequent treatment.

According to embodiments, the electronic device 215 or the console device 210 may display any information associated with a patient that has been previously stored to the electronic device 215 from querying the server 225. The information may be used to help match the patient to be treated to the patient that is brought to the medical device (e.g., an image of the patient, patient name, age, etc.).

According to embodiments, the software executable by the electronic device 215 for each medical device may be centralized on a single server dedicated to a physical location using one or more hardware virtualization techniques which enables for creating multiple virtual computers (i.e., software-defined computers) on a single physical computer. In this variation, the software may be separated from its dedicated physical computer. However, this variation would not require modification of the software as is but may require modification in how the software is deployed.

In alternative or additional embodiments, an additional electronic device (or software executed by either the electronic device 215 or the server 225) may encrypt any data that is sent from the electronic device 215 to the server 225 (and/or vice-versa), for example the “as-treated” data. Thus, the systems and methods may support full encryption of the data on the network(s).

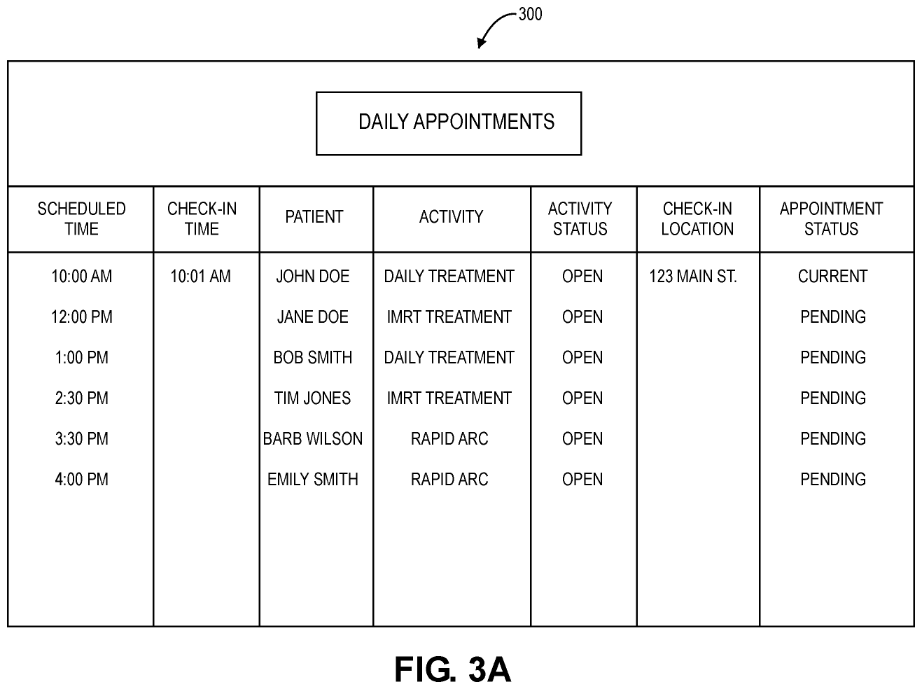

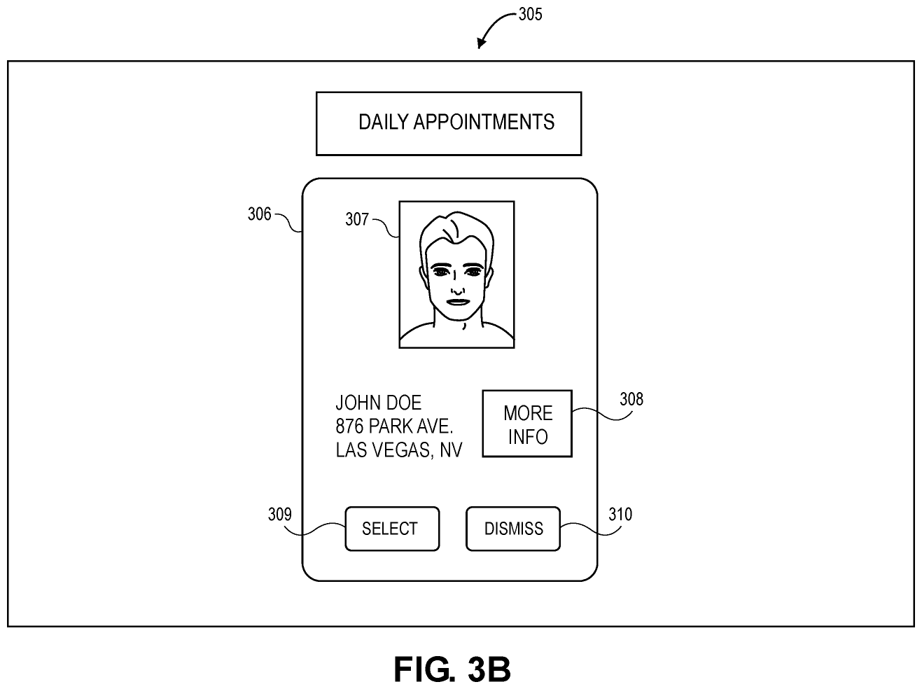

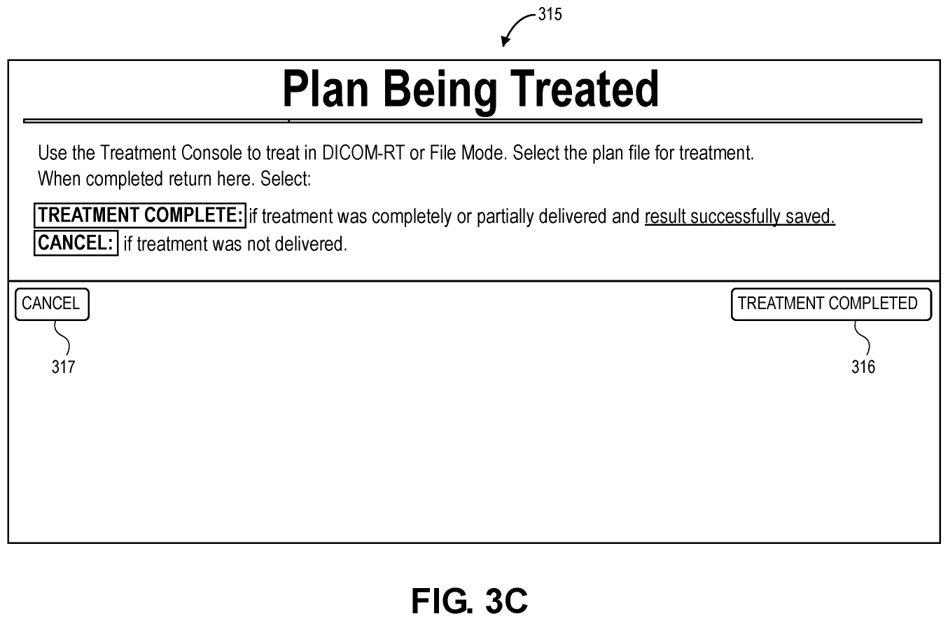

FIGS. 3A-3C illustrate example interfaces associated with the systems and methods. In particular, the interfaces may be displayed by the electronic device 115 as discussed with respect to FIG. 1B. It should be appreciated that the interfaces of FIGS. 3A-3C are exemplary and that the interfaces may include alternative or additional content. Further, it should be appreciated that the electronic device 115 may cause the interfaces of FIGS. 3A-3C to be displayed on a separate or auxiliary device that may be local to or remote from the electronic device 115. For example, the electronic device 115 may avail the interfaces via a website that may be accessed by a separate electronic device.

FIG. 3A illustrates an interface 300 that indicates a set of appointments for a set of patients that may be scheduled or set for a particular medical device on a particular day. Each of the appointments may list various information associated with that appointment, where the interface 300 may enable a user to check in a particular appointment. For example, an appointment 301 may be scheduled for 10:00 AM for patient John Doe to receive a daily treatment. Additionally, the interface 300 indicates that John Doe checked in at 10:01 AM at 123 Main St. and that the appointment is current with an activity status of “Open.”

FIG. 3B illustrates an interface 305 that the electronic device 115 may display in response to a user selecting one of the appointments listed in the interface 300 of FIG. 3A. As illustrated in FIG. 3B, the interface 305 may include a window 306 having various information associated with the selected appointment, including an image 307 of the patient, address information for the patient, a selection 308 for more information for the patient, a selection 309 to select the patient (e.g., to initiate treatment for the patient), and a selection 310 to dismiss the window 306 and return to a previous screen.

FIG. 3C illustrates an interface 315 that the electronic device 115 may display in association with a treatment being administered to a selected patient. The interface 315 may instruct the user to select a selection 316 (“Treatment Completed”) if the treatment was completely or partially delivered and a result successfully saved, or a selection 317 (“Cancel”) if the treatment was not delivered. In embodiments, in response to selection of the selection 316, the electronic device may store any “as-treated” data to additional non-volatile storage and delete the “as-treated” data from the temporary staging location, as discussed herein.

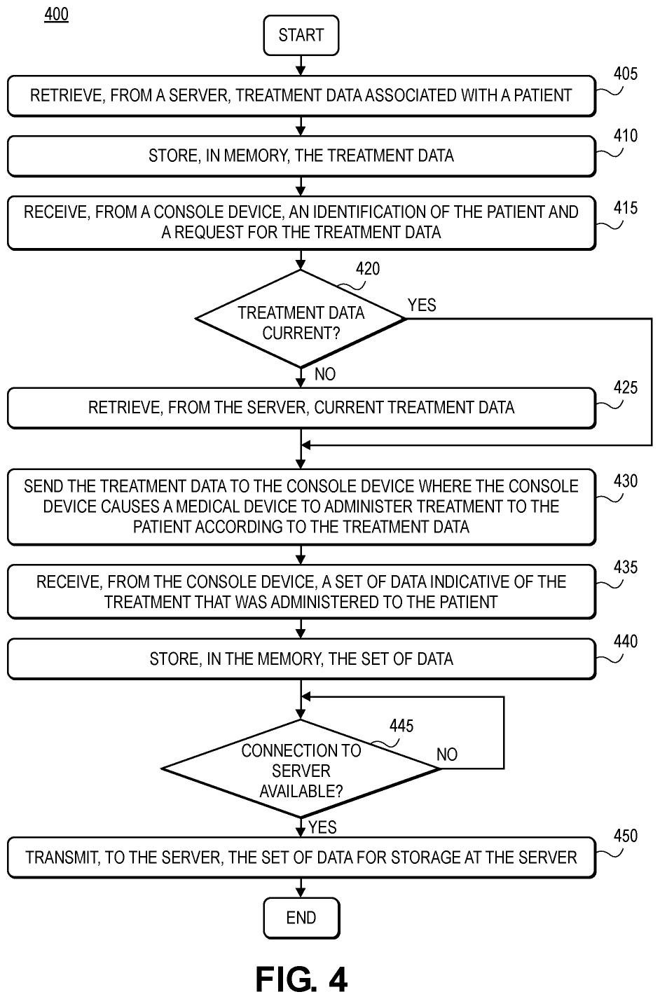

FIG. 4 depicts a block diagram of an example method 400 of managing treatment for patients. The method 400 may be facilitated by an electronic device (such as the electronic device 115, 215 as discussed with respect to FIGS. 1B and 2). In embodiments, the electronic device may be communicatively connected to a console device that may be communicatively connected to a medical device that administers treatment to the patients.

The method 400 may begin at block 405 in which the electronic device may retrieve, from a server, treatment data associated with a patient. In embodiments, the electronic device may retrieve the treatment data via a first network connection. Further, the electronic device may periodically retrieve, from the server at regular intervals, the treatment data associated with the patient. The electronic device may store (block 410), in memory, the treatment data, where the memory may be locally accessible by the electronic device.

The electronic device may receive (block 415), from a console device, an identification of the patient and a request for the treatment data. In embodiments, the electronic device may receive the identification and the request via a second network connection. Further, the first network connection may be a wide area network connection and the second network connection may be a local area network connection. The electronic device may determine (block 420) whether the treatment data is current In embodiments, the treatment data may be tagged with a unique identification (UID), and the electronic device may perform this determination by determining whether the treatment data tagged with the unique identification is stored in the memory.

If the electronic device determines that the treatment data is current (“YES”), processing may proceed to block 430. If the electronic device determines that the treatment data is not current (“NO”), the electronic device may retrieve (block 425), from the server, updated treatment data, and proceed to block 430.

At block 430, the electronic device may send the treatment data to the console device, where the console device causes a medical device to administer treatment to the patient according to the treatment data. After the treatment is administered, the electronic device may receive (block 435), from the console device, a set of data indicative of the treatment or therapy that was administered to the patient.

At block 440, the electronic device may store, in the memory, the set of data. Further, the electronic device may determine (block 445) whether there is an available connection to the server. If the connection to the server is unavailable (“NO”), the electronic device may repeat block 445 until a connection is available or until a timeout period ends. If the connection to the server is available (“YES”), the electronic device may transmit (block 450), to the server, the set of data for storage at the server. In embodiments, the electronic device may delete or overwrite the set of data stored in the memory assuming that the server successfully stores the set of data.

In embodiments, the electronic device may retrieve, from the server, patient information associated with the patient, and display, in a user interface (e.g., a user interface associated with the electronic device), at least a portion of the patient information. It should be appreciated that various types of patient information are envisioned. Further, it should be appreciated that the patient information may be displayed on or accessed via another electronic device that may be auxiliary to or remote from the electronic device.

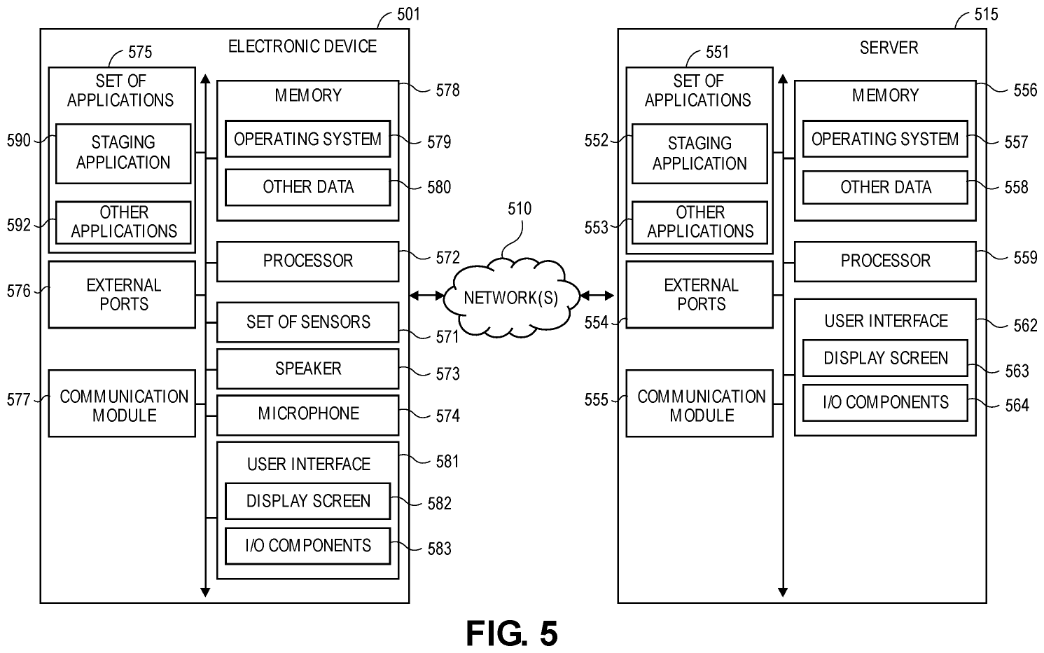

FIG. 5 illustrates a hardware diagram of an example electronic device 501 (e.g., one of the electronic devices 115, 215 as described with respect to FIGS. 1B and 2) and an example server 515 (e.g., one of the servers 125, 225 as described with respect to FIGS. 1B and 2), in which the functionalities as discussed herein may be implemented. It should be appreciated that the components of the electronic device 501 may also be present, in whole or in part, in the console device 110 and/or the medical device 105 as described with respect to FIG. 1B.

The electronic device 501 may include a processor 572 as well as a memory 578. The memory 578 may store an operating system 579 capable of facilitating the functionalities as discussed herein as well as a set of applications 575 (i.e., machine readable instructions). For example, one of the set of applications 575 may be a staging application 590, such as to enable facilitation and storage of treatment data among components. It should be appreciated that one or more other applications 592 are envisioned.

The processor 572 may interface with the memory 578 to execute the operating system 579 and the set of applications 575. According to some embodiments, the memory 578 may also store other data 580 that may include data associated with patients and treatments. The memory 578 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The electronic device 501 may further include a communication module 577 configured to communicate data via one or more networks 510. According to some embodiments, the communication module 577 may include one or more transceivers (e.g., WAN, WWAN, WLAN, and/or WPAN transceivers) functioning in accordance with IEEE standards, 3GPP standards, or other standards, and configured to receive and transmit data via one or more external ports 576.

The electronic device 501 may include a set of sensors 571 such as, for example, a location module (e.g., a GPS chip), an image sensor, an accelerometer, a clock, a gyroscope (i.e., an angular rate sensor), a compass, a yaw rate sensor, a tilt sensor, telematics sensors, and/or other sensors. The electronic device 501 may further include a user interface 581 configured to present information to a user and/or receive inputs from the user. As shown in FIG. 5, the user interface 581 may include a display screen 582 and I/O components 583 (e.g., ports, capacitive or resistive touch sensitive input panels, keys, buttons, lights, LEDs, and/or built in or external keyboard). Additionally, the electronic device 501 may include a speaker 573 configured to output audio data and a microphone 574 configured to detect audio.

In some embodiments, the electronic device 501 may perform the functionalities as discussed herein as part of a “cloud” network or may otherwise communicate with other hardware or software components within the cloud to send, retrieve, or otherwise analyze data.

As illustrated in FIG. 5, the electronic device 501 may communicate and interface with the server 515 via the network(s) 510. The server 515 may include a processor 559 as well as a memory 556. The memory 556 may store an operating system 557 capable of facilitating the functionalities as discussed herein as well as a set of applications 551 (i.e., machine readable instructions). For example, one of the set of applications 551 may be a staging application 552. It should be appreciated that one or more other applications 553 are envisioned.

The processor 559 may interface with the memory 556 to execute the operating system 557 and the set of applications 551. According to some embodiments, the memory 556 may also store other data 558 such as patient and treatment data. The memory 556 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The server 515 may further include a communication module 555 configured to communicate data via the one or more networks 510. According to some embodiments, the communication module 555 may include one or more transceivers (e.g., WAN, WWAN, WLAN, and/or WPAN transceivers) functioning in accordance with IEEE standards, 3GPP standards, or other standards, and configured to receive and transmit data via one or more external ports 554.

The server 515 may further include a user interface 562 configured to present information to a user and/or receive inputs from the user. As shown in FIG. 5, the user interface 562 may include a display screen 563 and I/O components 564 (e.g., ports, capacitive or resistive touch sensitive input panels, keys, buttons, lights, LEDs, external or built in keyboard). According to some embodiments, the user may access the server 515 via the user interface 562 to review information, make selections, and/or perform other functions.

In some embodiments, the server 515 may perform the functionalities as discussed herein as part of a “cloud” network or may otherwise communicate with other hardware or software components within the cloud to send, retrieve, or otherwise analyze data.

In general, a computer program product in accordance with an embodiment may include a computer usable storage medium (e.g., standard random access memory (RAM), an optical disc, a universal serial bus (USB) drive, or the like) having computer-readable program code embodied therein, wherein the computer-readable program code may be adapted to be executed by the processors 572, 559 (e.g., working in connection with the respective operating systems 579, 557) to facilitate the functions as described herein. In this regard, the program code may be implemented in any desired language, and may be implemented as machine code, assembly code, byte code, interpretable source code or the like (e.g., via Golang, Python, Scala, C, C++, Java, Actionscript, Objective-C, Javascript, CSS, XML). In some embodiments, the computer program product may be part of a cloud network of resources.

Although the following text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical.

What is claimed is:

1. A computer-implemented method in an electronic device of managing treatment for patients, the electronic device communicatively connected to a console device that is communicatively connected to a medical device that administers treatment to the patients, the computer-implemented method comprising:
retrieving, by a processor from a server via a first network connection, treatment data associated with a patient;
storing, in a memory of the electronic device, the treatment data associated with the patient;
receiving, by the processor from the console device via a second network connection, an identification of the patient and a request for the treatment data associated with the patient;
sending, by the processor to the console device via the second network connection, the treatment data, wherein the console device causes the medical device to administer the treatment to the patient according to the treatment data; and
after the medical device administers the treatment to the patient according to the treatment data:
receiving, by the processor from the console device via the second network connection, a set of data indicative of the treatment that was administered to the patient,
storing, in the memory, the set of data, and
transmitting, to the server, the set of data for storage at the server.

2. The computer-implemented method of claim 1, wherein the treatment data is tagged with a unique identification, and wherein sending the treatment data comprises:
determining, by the processor, that the treatment data tagged with the unique identification is stored in the memory; and
in response to determining that the treatment data tagged with the unique identification is stored in the memory, sending, by the processor to the console device via the second network connection, the treatment data that was stored in the memory.

3. The computer-implemented method of claim 1, wherein the treatment data is tagged with a unique identification, and wherein sending the treatment data comprises:
determining, by the processor, that the treatment data tagged with the unique identification is not stored in the memory; and
in response to determining that the treatment data tagged with the unique identification is not stored in the memory:
retrieving, by the processor from the server via the first network connection, current treatment data associated with the patient, and
sending, by the processor to the console device via the second network connection, the current treatment data.

4. The computer-implemented method of claim 1, wherein retrieving the treatment data associated with the patient comprises:
periodically retrieving, by the processor from the server via the first network connection at a regular interval, the treatment data associated with the patient.

5. The computer-implemented method of claim 1, wherein the first network connection is a wide area network connection and the second network connection is a local area network connection.

6. The computer-implemented method of claim 1, further comprising:
retrieving, by the processor from the server via the first network connection, patient information associated with the patient; and
displaying, in a user interface, at least a portion of the patient information.

7. The computer-implemented method of claim 1, wherein transmitting, to the server via the first network connection, the set of data for storage at the server comprises:
initially detecting that the server is unavailable via the first network connection,
subsequently detecting that the server is available via the first network connection, and
in response to detecting that the server is available via the first network connection, transmitting, to the server via the first network connection, the set of data that was stored in the memory, for storage at the server.

8. An electronic device for managing treatment for patients, the electronic device communicatively connected to a console device that is communicatively connected to a medical device that administers treatment to the patients, the electronic device comprising:
a transceiver configured to connect the electronic device to a server via a first network connection and to a console device via a second network connection;
a memory; and
a processor interfacing with the transceiver and the memory, and configured to:
retrieve, from a server via the transceiver, treatment data associated with a patient,
store, in the memory, the treatment data associated with the patient,
receive, from the console device via the transceiver, an identification of the patient and a request for the treatment data associated with the patient,
send, to the console device via the transceiver, the treatment data, wherein the console device causes the medical device to administer the treatment to the patient according to the treatment data, and
after the medical device administers the treatment to the patient according to the treatment data:
receive, from the console device via the transceiver, a set of data indicative of the treatment that was administered to the patient,
store, in the memory, the set of data, and
transmit, to the server via the transceiver, the set of data for storage at the server.

9. The electronic device of claim 8, wherein the treatment data is tagged with a unique identification, and wherein to send the treatment data, the processor is configured to:
determine that the treatment data tagged with a unique identification is stored in the memory, and
in response to determining that the treatment data tagged with the unique identification is stored in the memory, send, to the console device via the transceiver, the treatment data that was stored in the memory.

10. The electronic device of claim 8, wherein the treatment data is tagged with a unique identification, and wherein to send the treatment data, the processor is configured to:
determine that the treatment data tagged with a unique identification is not stored in the memory, and
in response to determining that the treatment data tagged with the unique identification is not stored in the memory:
retrieve, from the server via the transceiver, current treatment data associated with the patient, and
send, to the console device via the second network connection, the current treatment data.

11. The electronic device of claim 8, wherein the processor periodically retrieves, from the server via the first network connection at a regular interval, the treatment data associated with the patient.

12. The electronic device of claim 8, wherein the first network connection is a wide area network connection and the second network connection is a local area network connection.

13. The electronic device of claim 8, wherein the processor is further configured to:
retrieve, from the server via the transceiver, patient information associated with the patient, and
cause a user interface to display at least a portion of the patient information.

14. The electronic device of claim 8, wherein to transmit, to the server via the transceiver, the set of data for storage at the server, the processor is configured to:
initially detect that the server is unavailable via the first network connection,
subsequently detect that the server is available via the first network connection, and
in response to detecting that the server is available via the first network connection, transmit, to the server via the transceiver, the set of data that was stored in the memory, for storage at the server.

15. A non-transitory computer-readable storage medium having stored thereon a set of instructions, executable by at least one processor of an electronic device, for managing treatment for patients, the electronic device communicatively connected to a console device that is communicatively connected to a medical device that administers treatment to the patients, the instructions comprising:
instructions for retrieving, from a server via a first network connection, treatment data associated with a patient;
instructions for storing, in a memory, the treatment data associated with the patient;
instructions for receiving, by a processor of the electronic device from the console device via a second network connection, an identification of the patient and a request for the treatment data associated with the patient;
instructions for sending, by the processor to the console device via the second network connection, the treatment data, wherein the console device causes the medical device to administer the treatment to the patient according to the treatment data; and
instructions for, after the medical device administers the treatment to the patient according to the treatment data:
receiving, from the console device via the second network connection, a set of data indicative of the treatment that was administered to the patient,
storing, in the memory, the set of data, and
transmitting, to the server, the set of data for storage at the server.

16. The non-transitory computer-readable storage medium of claim 15, wherein the treatment data is tagged with a unique identification, and wherein the instructions for sending the treatment data comprise:
instructions for determining that the treatment data tagged with the unique identification is stored in the memory; and
instructions for, in response to determining that the treatment data tagged with the unique identification is stored in the memory, sending, to the console device via the second network connection, the treatment data that was stored in the memory.

17. The non-transitory computer-readable storage medium of claim 15, wherein the treatment data is tagged with a unique identification, and wherein the instructions for sending the treatment data comprise:
instructions for determining that the treatment data tagged with the unique identification is not stored in the memory; and
instructions for, in response to determining that the treatment data tagged with the unique identification is not stored in the memory:
retrieving, from the server via the first network connection, current treatment data associated with the patient, and
sending, to the console device via the second network connection, the current treatment data.

18. The non-transitory computer-readable storage medium of claim 15, wherein the instructions for retrieving the treatment data associated with the patient comprise:
instructions for periodically retrieving, from the server via the first network connection at a regular interval, the treatment data associated with the patient.

19. The non-transitory computer-readable storage medium of claim 15, wherein the instructions further comprise:
instructions for retrieving, from the server via the first network connection, patient information associated with the patient; and
instructions for displaying, in a user interface, at least a portion of the patient information.

20. The non-transitory computer-readable storage medium of claim 15, wherein the instructions for transmitting, to the server via the first network connection, the set of data for storage at the server comprise:
instructions for initially detecting that the server is unavailable via the first network connection,
instructions for subsequently detecting that the server is available via the first network connection, and
instructions for, in response to detecting that the server is available via the first network connection, transmitting, to the server via the first network connection, the set of data that was stored in the memory, for storage at the server.
